(19) United States
(12) Patent Application Publication
(10) Pub. No.: US 2002/0183747 A1
(43) Pub. Date: Dec. 5, 2002

(54) Spinal fixation apparatus

(75) Inventors: Wei-Tai Jao, Hsin-Chu (TW); Chih-Ming Wu, Hsin-Chu (TW); Chen-Dao Shaio, Hsin-Chu (TW); Ben-Hwa Jang, Taipei (TW); Audy Choeo, Tao-Yuan Hsien (TW); Chien-Lin Liu, Taipei (TW); Hung-Yi Chen, Taipei (TW)

Correspondence Address: BACON & THOMAS, PLLC, 625 SLATERS LANE, FOURTH FLOOR, ALEXANDRIA, VA, 22314

(73) Assignee: Merries International Inc., Taipei (TW)

(21) Appl. No.: 10/154,800

(22) Filed: May 28, 2002

(30) Foreign Application Priority Data
May 30, 2001 (TW) 090208804

(51) Int. Cl. A61B 17/56

(52) U.S. Cl. 606/61

(57) ABSTRACT

A spinal fixation apparatus consists of a connection means, a plurality of implant elements, washers and fastening elements. The implant elements are fastened to chines of the spine, and then the connection means is coupled on the upper side of the implant elements. Then corresponding washers and fastening elements are sequentially assembled. Finally the fastening elements are tightened on the implant elements to fasten the injured or ill-inflicted chines to the splint securely to form an integrated member to achieve a secure anchoring effect.

### FIELD OF THE INVENTION

The present invention relates to a spinal fixation apparatus that is designed through mechanic analyses with shrunk component sizes and still has sufficient strength and fatigue durability, and is modularly designed to anchor an injured or ill-inflicted spine and is allowed to make across pitches and angular adjustments to splint the spine in an optimal condition to enhance mending and rehabilitating effect.

### BACKGROUND OF THE INVENTION

Many people living in modem time are used to sit or sleep at wrong postures, or do exercises the wrong ways, or run into accidents, and consequently suffer from spine injuries or ill-inflicted spine deterioration. The patients with spine injury usually have to take surgical operations to cure and mend the deformed spines. After surgery, the spine, whole or partial, usually is anchored by a splint for rehabilitation to help the patients restoring to normal conditions.

Design of the splint for human spine has to take into account many factors, such as ergonomics, structural strength, and the like. The splint for spine generally has to be implanted inside human body to mend the injured or ill-inflicted chines. Based on different physical sizes of the patients, the splint has to be adjusted across pitches and angularly to make the splint fit smugly with the chines to achieve desired anchoring effect.

In order to minimize inconvenience to patients' movements, the dimensions of the splint should be as small as possible, and protrusion from the chines should also be minimized. While trying to reduce the size of the splint, the strength and fatigue durability of the splint should not be compromised to prevent the splint from breaking down or rupture, and to avoid the patients from suffering additional injury.

In addition, human nerve systems are clustered around the rear side of the spine. Surgery for the spine and its surrounding areas is a very complex and delicate operation. To facilitate surgical operations, shorten operation time and enhance surgery safety, the splint for the spine and chines should be simplified with less number of components whenever possible.

Moreover, different patients have different injured or ill-inflicted spine areas or chines. Hence design of the splint should have the flexibility for adjustments across the pitches and angularly to satisfy requirements of different conditions.

### SUMMARY OF THE INVENTION

The primary object of the invention is to resolve aforesaid problems and concerns. The invention provides a spinal fixation apparatus that can effectively reduce the protrusive height from the spine.

Another object of the invention is to provide a spinal fixation apparatus with composing elements optimally designed through mechanic analyses to equip with sufficient strength and fatigue durability.

A further object of the invention is to provide a spinal fixation apparatus that is composed of less number of anchoring elements and adopts an assembling and anchoring method which is done sequentially from down to top to make surgical operations simpler.

Yet another object of the invention is to provide a spinal fixation apparatus that is adjustable across pitches and angularly.

To achieve the foregoing objects, the splint of the invention includes a connection means, a plurality of implant elements, washers, and fastening elements. The connection means connects the implant elements, washers and fastening elements. The elements are adjustable across pitches and angularly. The fastening elements can fasten the implant elements tightly to completely anchor the injured or ill-inflicted spine or chines.

The foregoing, as well as additional objects, features and advantages of the invention will be more readily apparent from the following detailed description, which proceeds with reference to the accompanying drawings.

### BRIEF DESCRIPTION OF THE DRAWINGS

FIG. 1 is an exploded perspective view of the invention.

FIG. 2 is an exploded sectional view of the invention.

FIG. 3 is a schematic sectional view of the invention.

FIG. 4 is a schematic view of a first embodiment of the invention.

FIG. 5 is an exploded view of another type of a connection means of the invention.

FIG. 6 is a fragmentary sectional view of another type of the connection means of the invention.

FIG. 7 is a schematic view of a second embodiment of the invention.

FIG. 8 is an exploded perspective view of another type of the implant element and fastening element of the invention.

FIG. 9 is an exploded sectional view of another type of the implant element and fastening element of the invention.

FIG. 10 is a schematic view of a third embodiment of the invention.

### DESCRIPTION OF THE PREFERRED EMBODIMENTS

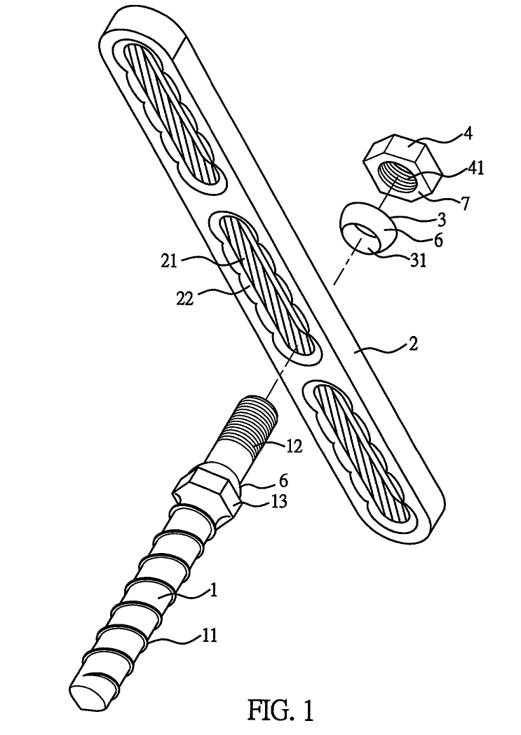

Referring to FIG. 1, the splint according to the invention consists of a plurality of implant elements 1, washers 3, fastening elements 4 and a connection means 2.

The implant elements 1, connection means 2, washers 3, and fastening elements 4 may be integrally formed or fabricated.

The implant elements 1 and connection means 2 are optimally designed through mechanic analyses to equip with sufficient strength and fatigue durability.

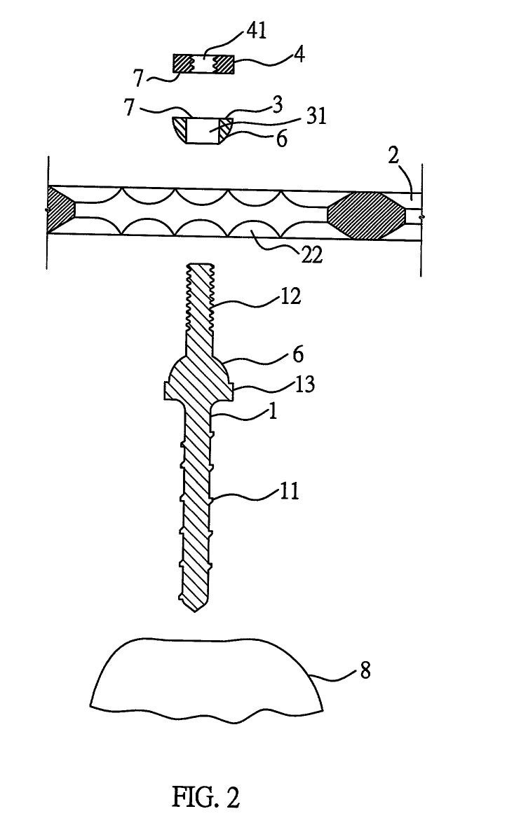

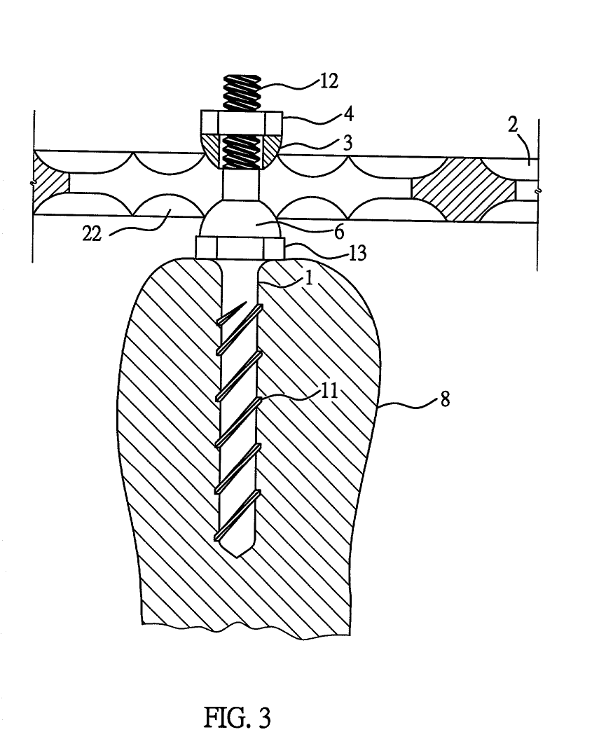

Referring to FIGS. 2 and 3, the implant element 1 is a rod having one end formed implant screw threads 11 for a selected length to fasten to a chine 8. The implant screw threads 11 may be formed in a conical shape to reduce fastening resistance. The implant element 1 has another end opposite to the implant screw threads 11 forming external fastening screw threads 12. There is a jutting ring 13 located between the implant screw threads 11 and external fastening screw threads 12. The jutting ring 13 has a plurality of tangent surfaces formed on the peripheral surface thereof for receiving force to turn the implant element 1. The jutting ring 13 has one side formed a spherical surface 6 facing the external fastening screw threads 12.

The connection means 2 is a narrow plank with two parallel planes and a plurality of slots 21 of a selected length formed longitudinally therein normal to the planes for coupling with the external fastening screw threads 12 of the implant element 1. The slot 21 and the peripheral edges of the external fastening screw threads 12 have a selected allowance formed therebetween to allow the implant element 1 making angular adjustments desired.

The slot 21 has two sides facing the two planes. At least one of the sides has a plurality of spherical concave surfaces 22 formed thereon to allow the implant element 1 running through the slot 21 to adjust position and angle according to the location and direction of the chine 8.

When both sides of the slot 21 have the spherical concave surfaces 22 formed thereon, the spherical concave surfaces 22 on both sides are corresponding to one another.

When only one side of the slot 21 has the spherical concave surfaces 22 formed thereon, the implant element 1 may have a bigger angular adjustment range.

When the connection means 2 is assembled, it can be bent in an arched shape according to the form of the chine 8.

The washer 3 has an aperture 31 of a selected size formed in the center thereof to couple with the external fastening screw threads 12 run through the slot 21. -The aperture 31 and the peripheral edges of the external fastening screw threads 12 have a selected allowance formed therebetween to allow the implant element 1 making angular adjustments desired. When one side of the connection means 2 is formed with the spherical surface 6, another side opposite to the spherical surface 6 may be formed with a fastening plane 7.

The spherical concave surfaces 22 of the connection means 2 has same spherical curvature as the spherical surface 6 of the implant element 1 and washer 3. Hence the implant element 1 may be adjusted angularly relative to the connection means 2 according to the location and direction of the chine 8.

The fastening element 4 is a strut element having a plurality of tangent surfaces to receive force for turning, and has a screw bore 41 in the center to engage with the external fastening screw threads 12 of the implant element 1. The fastening element 4 has a plane 7 formed on one side thereof facing the washer 3.

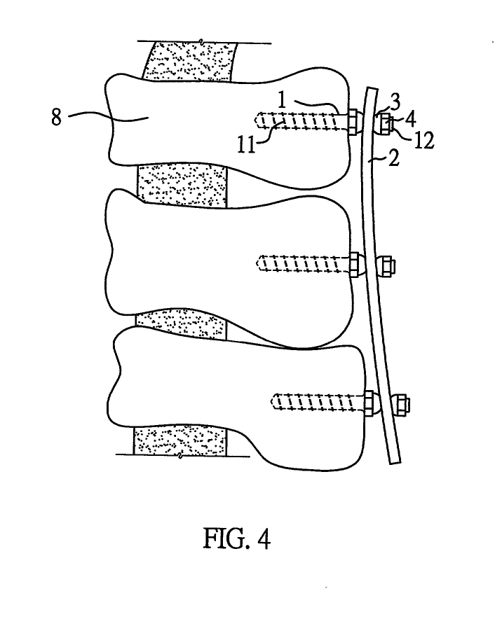

Referring to FIG. 4 for a first embodiment of the invention, a plurality of the implant elements 1 are screwed and fastened to chines 8 of the spine through the implant screw threads 11. Then sequentially assemble the connection means 2, washer 3 and fastening element 4 through the external fastening screw threads 12 of the implant elements 1. Finally the fastening element 4 is tightened to anchor the implant element 1, washer 3 and fastening element 4 through the connection means 2. Thus the injured or ill-inflicted chines 8 can be integrally fastened to the splint to achieve a secure anchoring.

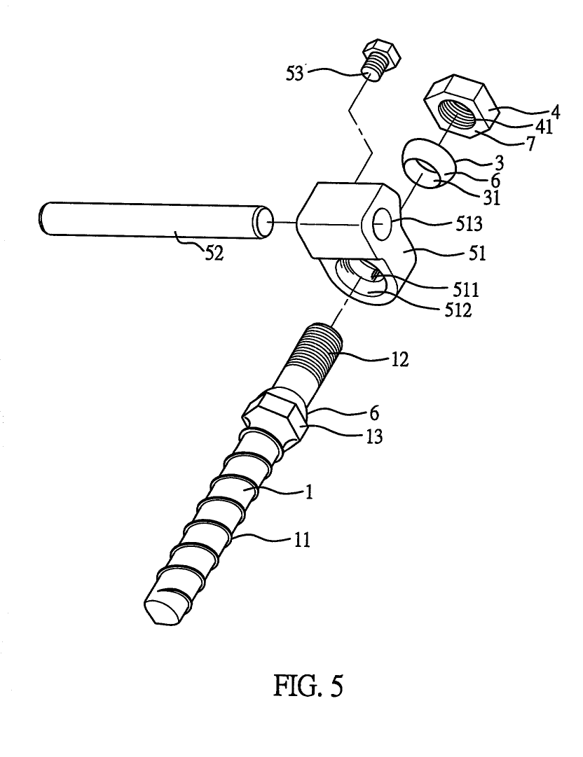

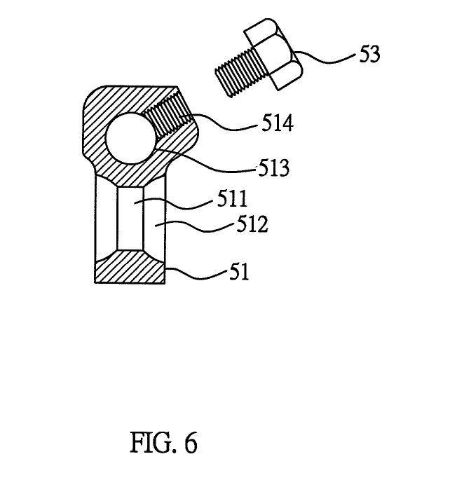

Referring to FIGS. 5 and 6 for another type of the connection means which consists of a plurality of connection elements 51, a rod 52 of an uniform cross section and a plurality of set screws 53.

The connection element 51 is an elongated plank having two parallel planes which have one end forming a first aperture 511 normal to the planes for coupling with the external fastening screw threads 12 of the implant element 1. The first aperture 511 and the peripheral edges of the external fastening screw threads 12 have a selected allowance formed therebetween to allow the implant element 1 making angular adjustments desired. The first aperture 511 has two ends facing the parallel planes and forming respectively a spherical and annular concave rim 512. The connection element 51 has another end opposing to the first aperture 511 and forming a second aperture 513 therein in parallel with the parallel planes. The second aperture 513 is bordered with a jutting side which has another plane with a set screw hole 514 formed therein to run through the another plane to communicate with the second aperture 513.

The spherical and annular concave rim 512 has the same spherical curvature as the spherical surfaces 6 of the implant element 1 and washer 3. Hence the implant element 1 may be adjusted angularly relative to the connection element 51 according to the location and direction of the chine 8.

The rod 52 of an uniform cross section may be modularly designed with different lengths to suit different pitches of the chines 8.

The set screw 53 is to engage with the set screw hole 514 for anchoring the rod 52 in the second aperture 513 on the connection element 5 1.

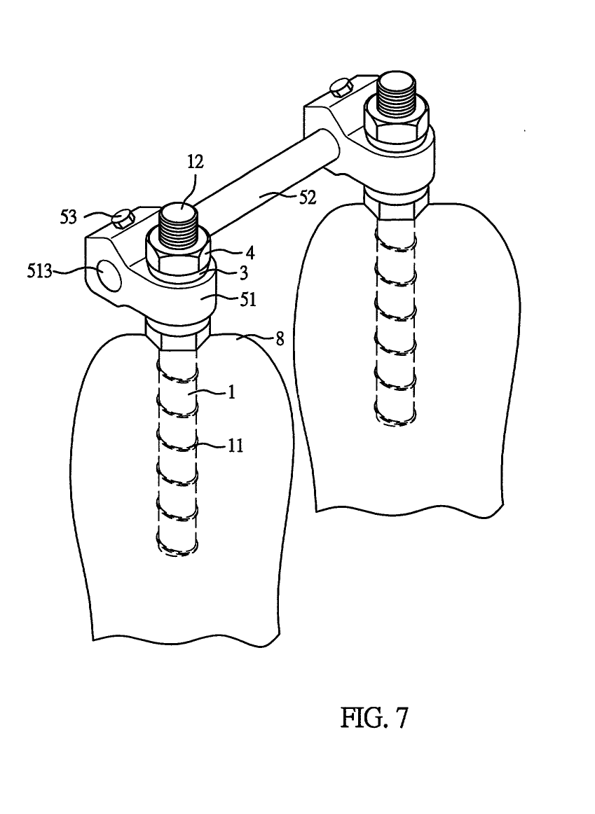

Referring to FIG. 7 for a second embodiment of the invention, a plurality of the implant elements 1 are screwed and fastened to chines 8 of the spine through the implant screw threads 11. Then sequentially assemble the connection element 51, washer 3 and fastening element 4 through the external fastening screw thread 12 of the implant element 1. Finally the fastening element 4 and the set screw 53 are tightened to anchor the implant element 1, connection element 51, washer 3 and fastening element 4 through the rod 52. Thus the injured or ill-inflicted chines 8 can be integrally fastened to the splint to achieve a secure anchoring.

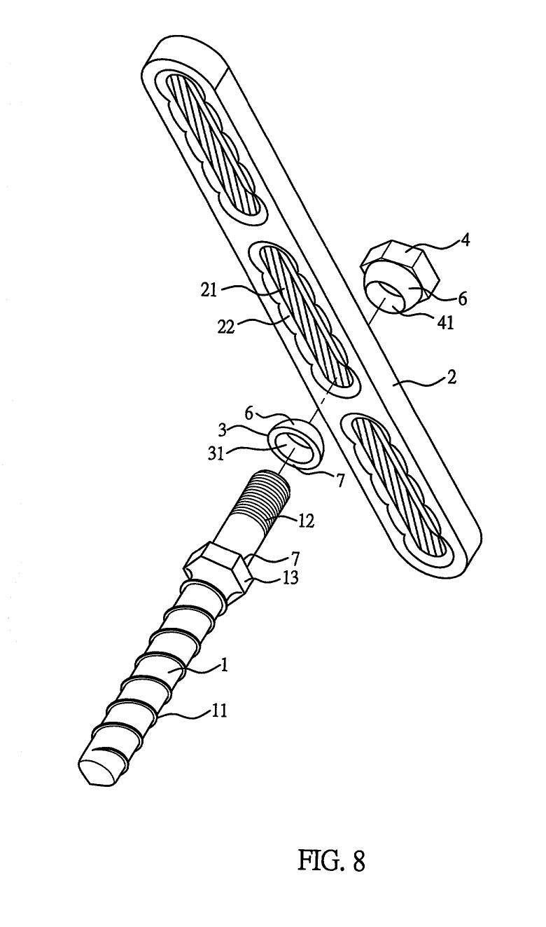

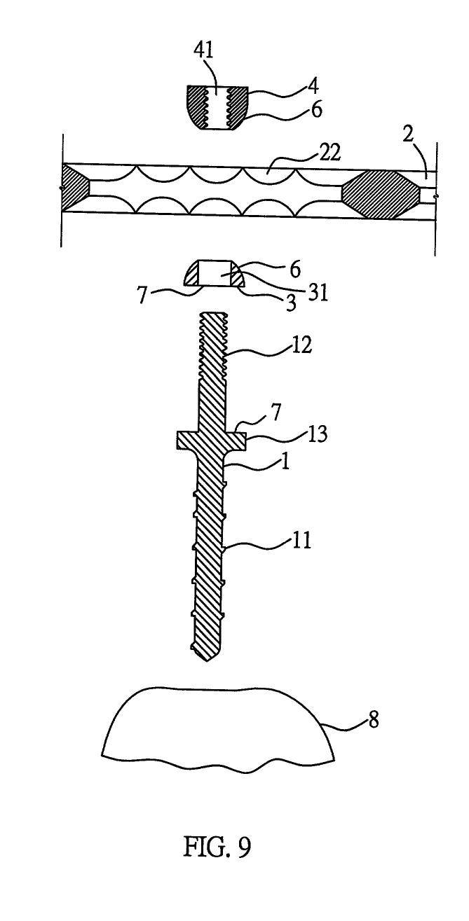

Referring to FIGS. 8 and 9 for another type of the implant element and fastening element, the implant element 1 is a rod having one end forming implant screw threads 11 for a selected length to fasten to the chine 8. The implant screw threads 11 may be formed in a conical shape to reduce fastening resistance. The implant element 1 has another end opposite to the implant screw threads 11 forming external fastening screw threads 12 for a selected length. There is a jutting ring 13 located between the implant screw threads 11 and external fastening screw threads 12. The jutting ring 13 has a plurality of tangent surfaces formed on the peripheral surface thereof for receiving force to turn the implant element 1. The jutting ring 13 has one side formed a fastening plane 7 facing the external fastening screw threads 12.

The fastening element 4 is a strut element having a plurality of tangent surfaces to receive force for turning, and has a screw bore 41 in the center to engage with the external fastening screw threads 12 of the implant element 1. The fastening element 4 has a spherical surface 6 formed on one side facing the connection means 2.

The spherical surface 6 of the fastening element 4 has same spherical curvature as the spherical concave surfaces 22 of the connection means 2. Hence the implant element 1 may be adjusted angularly relative to the connection means 2 according to the location and direction of the chine 8.

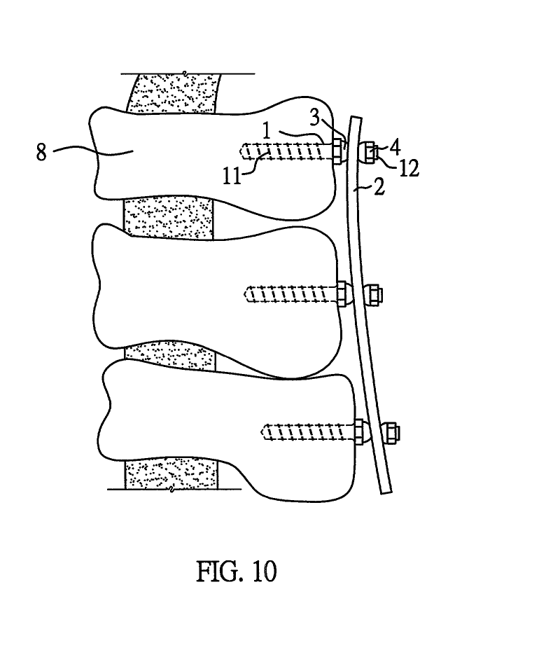

Referring to FIG. 10 for a third embodiment of the invention, a plurality of the implant elements 1 are screwed and fastened to chines 8 of the spine through the implant screw threads 11. Then sequentially assemble the washer 3, connection means 2 and fastening element 4 through the external fastening screw threads 12 of the implant element 1. Finally the fastening element 4 is tightened to anchor the implant element 1, washer 3 and fastening element 4 through the connection means 2. Thus the injured or ill-inflicted chines 8 can be integrally fastened to the splint to achieve a secure anchoring.

What is claimed is:

1. A spinal fixation apparatus, comprising:
a plurality of implant elements each being a rod and having one end formed implant screw threads for a selected length and another end opposite to the implant screw threads formed external fastening screw threads for another selected length, and a jutting ring located between the implant screw threads and the external fastening screw threads, the jutting ring having one side formed a spherical surface facing the external fastening screw threads;
a connection means engageable with the external fastening screw threads of the implant elements;
a plurality of washers located on an upper side of the connection means each having an aperture of a selected size formed in the center thereof for coupling with the external fastening screw threads of the implant element, a spherical surface formed on one side thereof facing the connection means and a fastening plane formed on another side thereof opposite to the spherical surface; and
a plurality of fastening elements each being a strut and having a screw bore in the center thereof to engage with the external fastening screw threads of the implant element, and a plane formed on one side thereof facing the washer;
wherein a plurality of the implant elements are screwed and fastened to chines of a spine, and the connection means, the washers and the fastening elements are sequentially assembled, and the fastening elements are tightened on the implant elements to fasten the injured or ill-inflicted chines to the splint to form an integrated member to achieve a secure anchoring.

2. The spinal fixation apparatus of claim 1, wherein the implant elements, the connection means, the washers and the fastening elements are integrally formed or fabricated.

3. The spinal fixation apparatus of claim 1, wherein the implant elements and the connection means are optimally designed through mechanic analyses.

4. The spinal fixation apparatus of claim 1, wherein the implant screw threads of the implant elements are formed in a conical shape.

5. The spinal fixation apparatus of claim 1, wherein the jutting ring of the implant element has a plurality of tangent surfaces formed on the peripheral surface thereof.

6. The spinal fixation apparatus of claim 1, wherein the connection means is a narrow plank with two parallel planes and has a plurality of slots with a selected length formed longitudinally therein normal to the planes for coupling with the external fastening screw threads of the implant elements, each slot having two sides facing the two planes, at least one of the sides having a plurality of spherical concave surfaces formed thereon.

7. The spinal fixation apparatus of claim 6, wherein the connection means is formed in an arched shape to match a spine.

8. The spinal fixation apparatus of claim 6, wherein both sides of the slot have the spherical concave surfaces formed thereon, the spherical concave surfaces on both sides being corresponding to one another.

9. The spinal fixation apparatus of claim 6, wherein the slot is coupled with the external fastening screw threads of the implant element, and has a selected allowance formed between the peripheral side thereof and the external fastening screw threads.

10. The spinal fixation apparatus of claim 6, wherein the spherical concave surfaces on the slot have same spherical curvature as the spherical surface of the implant elements and the washers.

11. The spinal fixation apparatus of claim 1, wherein each fastening element has a plurality of tangent surfaces formed on the peripheral surface thereof.

12. The spinal fixation apparatus of claim 1, wherein the connection means includes:
a plurality connection elements each being an elongated plank having two parallel planes which have one end forming a first aperture normal to the planes for coupling with the external fastening screw threads of the implant elements, the first aperture having two ends facing the parallel planes and forming respectively a spherical and annular concave rim, each connection element having another end opposing to the first aperture and forming a second aperture therein in parallel with the parallel planes, the second aperture being bordered with a jutting side which has another plane with a set screw hole formed therein to run through the another plane to communicate with the second aperture
a rod of an uniform cross section running through the second aperture of the connection element; and
a plurality of set screws each being engageable with the set screw hole of the connection element.

13. The spinal fixation apparatus of claim 12, wherein the first aperture is coupled with the external fastening screw threads of the implant element, and has a peripheral side forming a selected allowance with the external fastening screw threads.

14. The spinal fixation apparatus of claim 12, wherein the spherical and annular concave rim of the first aperture has same spherical curvature as the spherical surface of the implant elements and the washers.

15. The spinal fixation apparatus of claim 12, wherein the rod is replaceable and has a selected length and being modularly designed.

16. A spinal fixation apparatus, comprising:
a plurality of implant elements each being a rod and having one end formed implant screw threads for a selected length and another end opposite to the implant screw threads formed external fastening screw threads for another selected length, and a jutting ring located between the implant screw threads and the external fastening screw threads, the jutting ring having one side formed a fastening plane facing the external fastening screw threads;
a plurality of washers each having an aperture of a selected size formed in the center thereof for coupling with the external fastening screw threads of the implant elements, a fastening plane formed on one side thereof facing the implant element, and a spherical surface formed on another side thereof opposite to the fastening plane;
a connection means engageable with the external fastening screw threads of the implant elements; and
a plurality of fastening elements located on an upper side of the connection means each being a strut and having a screw bore in the center thereof to engage with the external fastening screw threads of the implant elements, and a spherical surface formed on one side thereof facing the connection means;
wherein a plurality of the implant elements are screwed and fastened to chines of a spine, and the washers, the connection means, and the fastening elements are sequentially assembled, and the fastening elements are tightened on the implant elements to fasten the injured or ill-inflicted chines to the splint to form an integrated member to achieve a secure anchoring.

17. The spinal fixation apparatus of claim 16, wherein the implant elements, the washers, the connection means, and the fastening elements are integrally formed or fabricated.

18. The spinal fixation apparatus of claim 16, wherein the implant elements and the connection means are optimally designed through mechanic analyses.

19. The spinal fixation apparatus of claim 16, wherein the implant screw threads of the implant element are formed in a conical shape.

20. The spinal fixation apparatus of claim 16, wherein the jutting ring of the implant element has a plurality of tangent surfaces formed on the peripheral surface thereof.

21. The spinal fixation apparatus of claim 16, wherein each of the fastening elements has a plurality of tangent surfaces formed on the peripheral surface thereof.
